(19) United States
(12) Patent Application Publication
(10) Pub. No.: US 2003/0107814 A1
(43) Pub. Date: Jun. 12, 2003

(54) Method and apparatus for improving the dynamic range and accuracy of a Shack-Hartmann wavefront sensor

(76) Inventor: Griffith E. Altmann, Webster, NY (US)

Correspondence Address: William Greener, Law Department, Bausch & Lomb Incorporated, One Bausch & Lomb Place, Rochester, NY, 14604, US

(21) Appl. No.: 10/013,565

(22) Filed: Dec. 11, 2001

(51) Int. Cl. G02B 27/10; A61B 3/00; G02B 27/14

(52) U.S. Cl. 359/619; 359/637

(57) ABSTRACT

A Shack-Hartmann aberrometer for measuring ophthalmic wavefront aberrations having improved dynamic range is described. In one embodiment, a single lenslet only of the wavefront sensor microlens array is optically or physically altered. In another embodiment, a sub-array of immediately adjacent lenslets are altered. In an alternative embodiment, preferably only two non-adjacent lenslets are altered. The alteration provides for identification of the correspondence between spot images of the unknown wavefront formed by the microlens array and the respective lenslets forming the spot images. Once all the spot images are correctly identified, analysis of the incoming wavefront is simplified even when the magnitude of aberration is increased. Thus, the dynamic range and accuracy of the Shack-Hartmann aberrometer are no longer limited by the dimensions and optical properties of the lenslets.

### BACKGROUND OF THE INVENTION

1. Field of the Invention

The present invention is directed to the field of wavefront sensing and, more particularly, to a method and apparatus for improving the dynamic range and accuracy of a Shack-Hartmann wavefront sensor.

2. Description of Related Art

A typical Shack-Hartmann wavefront sensor includes, among other components, a two-dimensional array of identical positive-power lenlets (microlens array) for imaging an aberrated wavefront. Although the invention described herein is applicable to all devices that measure wavefront with a Shack-Hartmann wavefront sensor, the applications and description appearing herein will refer to a Shack-Hartmann wavefront sensor used for ophthalmic applications. However, the invention is not so limited. FIG. 1 is an optical schematic of a typical Shack-Hartmann type aberrometer. Briefly, a light source 12 illuminates a subject's eye 50 while the subject fixates on a target 28. A focus error adjusting system 30 is used to correct refractive errors. Scattered/reflected light from the eye's retina, R, is directed to both a pupil camera 26 to aid in pupil alignment, and to a microlens array 18 that images the reflected light into an array of spots on a sensor 22 associated with a wavefront camera 20. The microlens array 18 and the image plane of the camera 20 are parallel to one another and spaced apart by the focal length, f, of the lenslets. When a plane wave passes through the lenslet array 18, the original wavefront is broken into smaller wavefronts that come to focus on a detector in the image plane of the camera. Each focused spot is located along the optical axis of its respective lenslet. If the wavefront is not planar, then some or all of the focused spots do not get imaged on the optical axis of the respective lenslet, but are offset. It is this positional difference that is used to evaluate the incoming wavefront.

The accuracy of the wavefront measurement with the Shack-Hartmann sensor lies in knowing which image spots correspond to which lenslets of the array. Since spots are, for the most part, indistinguishable, it is typically assumed that a spot from a particular lenslet can only be located within the area of interest of the lenslet, which is defined by the dimensions of the lenslet. However, this restriction limits the dynamic range of the wavefront sensor; i.e., the maximum wavefront slope that can be measured per lenslet. In addition, a wavefront with excessive aberrations may be incorrectly measured if some or all of the spot images are attributed to the wrong lenlets, resulting in erroneous position calculations.

Accordingly, the inventor has recognized a need for apparatus and methods to accurately locate the spot images and the corresponding lenslets that form the images in order to increase the accuracy of a Shack-Hartmann wavefront sensor measurement, and to improve the dynamic range of a Shack-Hartmann wavefront sensor. Moreover, there is a recognized need for a Shack-Hartmann wavefront sensor that can effectively measure larger amounts of aberration, for example, eyes with keratoconus and other pathologies and post LASIK or PRK eyes. These and other advantages of the present invention will become more apparent in view of the drawings and detailed description presented below.

### SUMMARY OF THE INVENTION

The invention is directed to an improved Shack-Hartmann aberrometer in which the improvement provides the aberrometer with a greater dynamic range, that is, the ability to measure a more aberrated wavefront without sacrificing sensitivity or accuracy of wavefront slope measurements. According to an embodiment of the invention, an improvement for a Shack-Hartmann wavefront sensing device that utilizes a microlens array for imaging the aberrated wavefront into a series of spot images on a detector resides in altering a single lenslet only of the microlens array to provide an identification of the spot images corresponding to their respective lenslets. In preferred aspects of the embodiment, an optical characteristic of the single lenslet or a physical characteristic of the single lenslet, or both, is altered such that that lenslet either will not form a spot image, the lenslet will form a spot image that is brighter or dimmer (contains more or less energy, respectively) than the surrounding spot images formed by their respective unaltered lenslets, or the spot image will be of substantially different size, shape and/or character than adjacent spot images.

In another embodiment, the invention is more broadly directed to a microlens array used in a Shack-Hartmann wavefront sensor wherein a single lenslet only of the array is altered either physically, optically, or in combination, or otherwise, to identify the presence or absence of a spot image corresponding to the single, altered lenslet and the spot images corresponding to the respective non-altered lenslets of the array. In an advantageous aspect of the foregoing embodiments, a central lenslet of the array is the altered lenslet.

In a likewise manner, a sub-array region of lenslets (i.e., a sub array of immediately adjacent lenslets), preferably a central sub-array region, may be altered to form, e.g., a single lenslet with a size equivalent to the sub-array size.

In alternative aspects related to the embodiments referred to above, more than a single lenslet but less than all of the lenslets of the array, and in this aspect preferably only two, non-adjacent lenslets, could be altered. This structural aspect of the invention offers the benefit of redundancy in the search and identification of the correspondence between spot images and the lenslets forming the respective images.

In a further embodiment, the invention is directed to a method for improving the dynamic range of a Shack-Hartmann aberrometer by identifying a correspondence between each of a plurality of spot images of an aberrated wavefront and respective lenslets of a microlens array that form the respective spot images. This embodiment preferably includes the steps of altering a single lenslet only of the microlens array, establishing the presence or absence of a spot image corresponding to the altered lenslet, and identifying therefrom the remaining spot images corresponding to the respective lenslets that formed the spot images. The lenslet alteration is preferably in the form of an optical or physical alteration of the single lenslet such that a spot image is not formed or, alternatively, a spot image is formed which has a different size, shape, intensity, and/or other identifying characteristic, than adjacent spot images formed by respective lenslets of the array. Alternatively, a related method for improving the dynamic range of a Shack-Hartmann aberrometer would involve altering more than a single lenslet but fewer than all of the lenslets of the array, and preferably only two, non-adjacent ones of the lenslets.

These and other objects of the present invention will become more readily apparent from the detailed description to follow. However, it should be understood that the detailed description and specific examples, while indicating the preferred embodiments of the invention, are given by way of illustration only, since various changes and modifications within the spirit and scope of the invention will become apparent to those skilled in the art based upon the descriptions and drawings herein and the appended claims.

### BRIEF DESCRIPTION OF THE DRAWINGS

FIG. 1 is an optical schematic of a typical Shack-Hartmann aberrometer used in conjunction with an embodiment of the invention;

FIG. 2a is a cross-sectional side-view schematic of a 3×3 microlens array having an opaque central lenslet according to an embodiment of the invention;

FIG. 2b is a schematic representation of a detector surface showing the positions of the spot images formed by the lenslets of the array in FIG. 2a;

FIG. 2c is a line drawing representing the intensity of the spot images in FIG. 2b across the center row from the left column to the right column;

FIG. 3a is a schematic front plan view of a microlens array according to an embodiment of the invention;

FIG. 3b is a schematic representation of a detector surface showing the positions of the spot images formed by the respective lenslets of the array of FIG. 3a;

FIG. 3c is a line drawing representing the intensity of the spot images in FIG. 3b; and

FIG. 4 is a schematic front plan view of a microlens array according to another embodiment of the invention.

### DETAIL DESCRIPTION OF A PREFERRED EMBODIMENT OF THE INVENTION

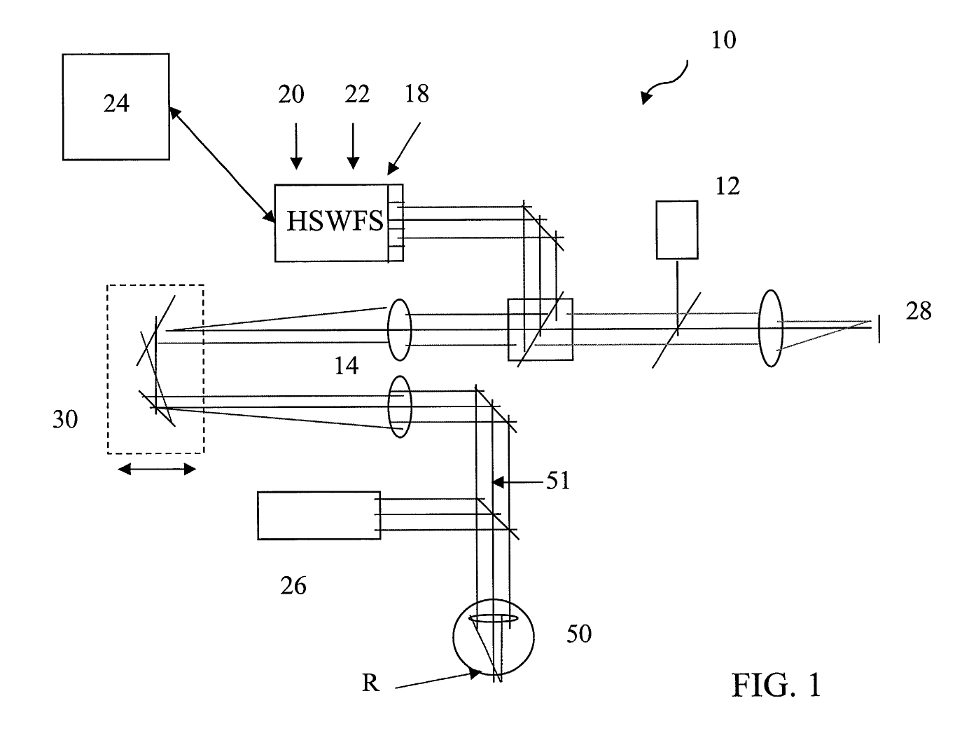

FIG. 1 shows an optical diagram of a Shack-Hartmann aberrometer 10. In general terms describing wavefront sensor operation, a patient's eye 50 is properly aligned with the measurement axis 51 of the aberrometer with the help of a fixation target 28 and an alignment camera 26, typically a pupil camera. The retina, R, of the eye is illuminated by light from a source 12 such as a 780 nm laser diode, for example, or other appropriate wavelength source, and the light is focused on the retina by an optical trombone system 30 and imaging lenses 14. The trombone system (or an alternate optical focusing system known in the art) is used to compensate for the simple near- or far-sightedness in the eye and also sharpens the focus of the image spots formed on a detector 22, resulting in more accurate wavefront measurement. The interested reader is referred to International Publication WO 01/28408 for a detailed description of the optical trombone system. This publication is incorporated herein by reference in its entirety to the extent permitted by applicable patent rules and laws. Reflected light from the retina passes out through the eye's optical system and on to the detector 22. In the Shack-Hartmann system, which currently is the dominant ophthalmic device methodology for diagnostic wavefront measurement, the reflected light is focused by a lenslet array 18 into aerial images on the detector 22 and displayed by a sensor camera 20. Image centroids are calculated and wavefront slope data is obtained from image displacement information using a processing system 24 which includes a P.C. and appropriate software for also calculating the aberration data, for command and control of aberrometer components, for data transfer, and for other various calculations using the wavefront information. The information is processed and typically fit to Zernike polynomials to output the aberration coefficient measurements. These coefficients can then be used in the design of corrective lenses, ablation algorithms, and in other ophthalmic applications known to those skilled in the art.

Current algorithms used in wavefront sensors for determining wavefront aberrations require that each image spot formed by a lenslet of a microlens array occur inside the area of the respective lenslet. This means that the maximum measurable wavefront slope (aberration) is limited by the size of the lenslet and its focal length. Assuming the entire spot must be located inside the area of the lenslet, the maximum measurable slope per lenslet is given by the following equation: tan(ₘₐₓ)=(d−s)/(2f), where ₘₐₓ is the maximum measurable slope, d is the width of the lenslet, s is the width or diameter of the spot, and f is the focal length of the lenslet. Thus the dynamic range of the instrument is limited by the size and focal length of the lenslets. Unfortunately increasing the size of the lenslets reduces the sampling and subsequently reduces the accuracy of the wavefront reconstruction. Advantageously, the invention eliminates these limitations by marking a particular lenslet such that spot image identification and lenslet correspondence are straightforward. If the spot images are uniquely identified, they can be determined to correspond to their respective lenslets even if they lie outside of the area of the lenslet. As a general rule, the sensitivity and/or accuracy of wavefront slope measurement are sacrificed at the expense of increased dynamic range. The minimum measurable slope per lenslet is given by the following equation: tan(ₘᵢₙ)=(minimum measurable displacement on the detector)/f. The minimum measurable displacement on the detector depends primarily upon the detector characteristics (i.e. the pixel dimensions and quality of the electronics). Thus, increasing the focal length of the lenslet improves the sensitivity of slope measurement, but reduces the maximum measurable slope.

According to the instant invention, neither sensitivity nor accuracy is sacrificed while dynamic range is improved. A basic assumption adhered to herein and in accordance with the prior art is that the wavefront being measured is assumed to be smooth and continuous; in other words, the spot images do not cross.

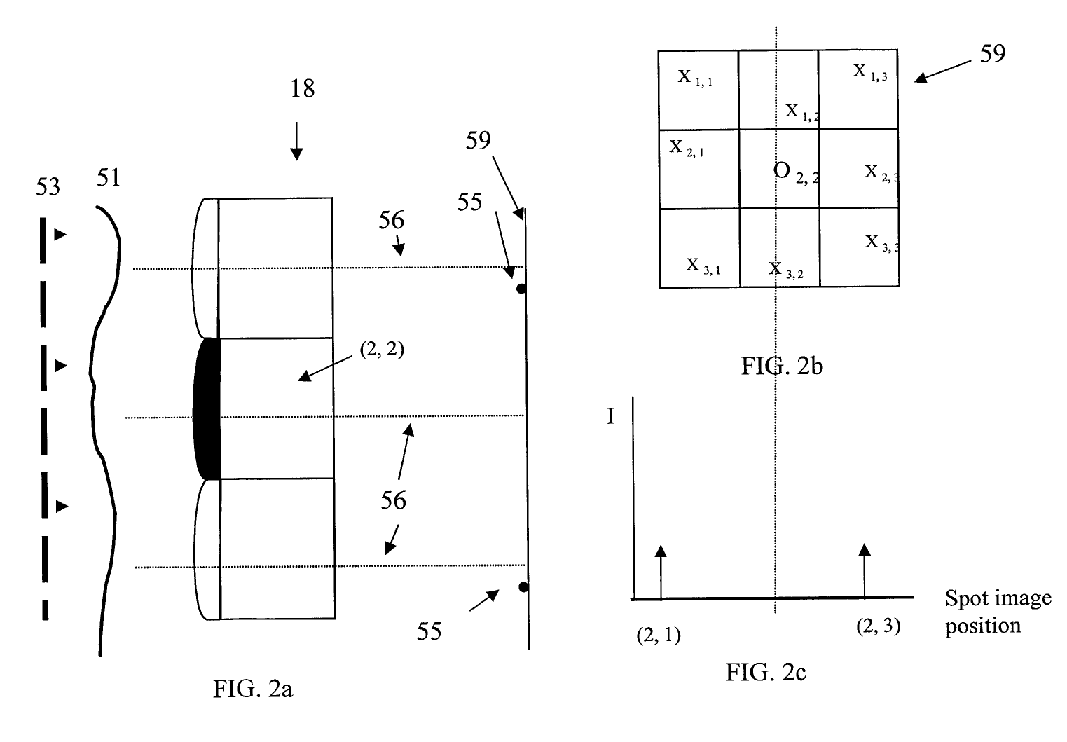

FIG. 2a is a schematic side-view of a microlens array 18 according to an embodiment of the invention. For the sake of description, the microlens array is a 3×3 lenslet array. As shown in a preferred embodiment, the center lenslet (2,2) is blackened to make it opaque to the light constituting the aberrated wavefront 51 to be measured. A center lenslet, or central lenslet sub-array, is preferred because the unknown wavefront is likely to be centered with respect to the microlens array. In addition, the center of the wavefront is often the least aberrated portion of the wavefront.

Typically, a reference or plane wavefront 53 incident on the microlens array would be imaged as an array of point images 55 on a detector 59 at locations on the optical axes 56 of the lenslets. This information is then stored in a CPU 24 of the aberrometer 10 (FIG. 1). As show in FIG. 2b, the aberrated wavefront 51 imaged by the microlens array 18 is represented by “x's” in cell areas on the detector 59 corresponding to each of the lenslet areas. The displacement of each of the spot images from the corresponding centered, plane reference wavefront image positions on the detector is proportional to the local slope of the wavefront at each lenslet location. These displacement values are then used to calculate the Zernike coefficients representing the higher order aberrations of the wavefront. As shown in FIGS. 2a, b and c, central lenslet (2,2) is optically altered with ink, or another suitable obstruction, to be opaque to the wavelength of the aberrated wavefront such that no spot image is formed in image cell (2,2) of the detector in FIG. 2b. The absence of a spot image corresponding to the altered lenslet (2,2) precisely identifies the correspondence of the spot images that are formed with their respective lenslets. Thus the spot image x_{1, 2} above the anticipated position of the [missing] image from the altered lenslet will correspond to lenslet (1, 2); the image x_{2, 3} immediately to the right will correspond to lenslet (2, 3); and so on. FIG. 2c diagrammatically shows a scan of the intensity of the spot images across the middle row of the detector. As expected, locations corresponding to lenslet cells 2,2 and 2,3 are present while no light intensity appears in the cell area 2,2 due to the opacity of that corresponding lenslet. In an alternative aspect, the lenslet could be coated to dim the spot image with respect to the other spots.

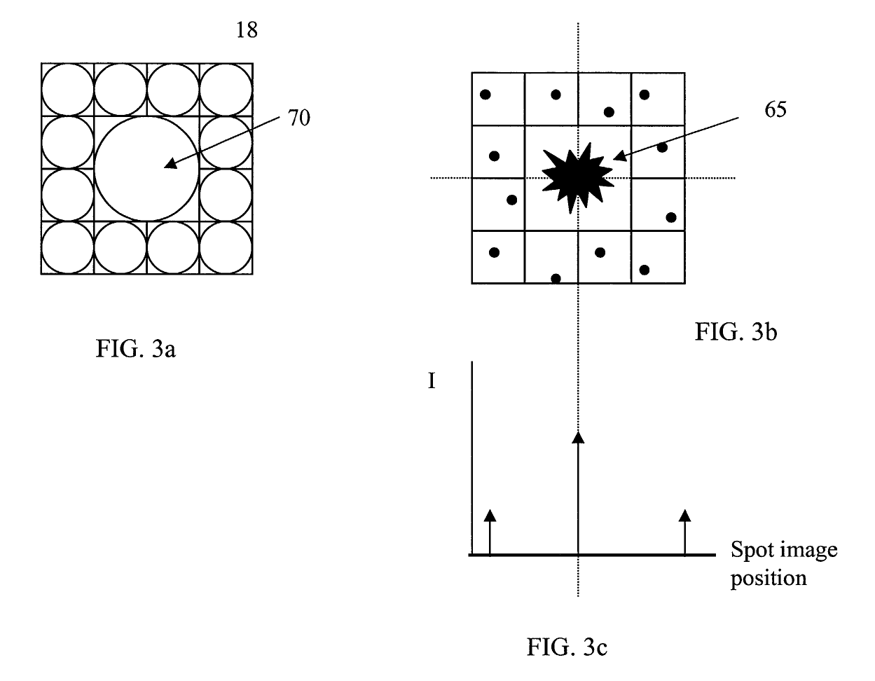

FIG. 3a shows a microlens array 18 according to an embodiment of the invention in which the lenslet array is physically altered, in contrast to the optical alteration shown in FIG. 2a. In FIG. 3a, a representative 4×4 microlens array is physically altered such that the central four-lenslet sub-array is replaced by a single, larger lenslet 70. The effect here, as shown in FIGS. 3b and 3c, is that the spot image 65 corresponding to the single, physically altered lenslet 70 has four times the intensity of the spot images corresponding to the non-altered lenslets. In addition, the diameter of the spot resulting from the altered, 4×larger lenslet will be roughly 4×smaller than that of spots resulting from the unaltered, smaller lenslets, assuming the wavefront is appropriately sampled such that each sub-aperture of the wavefront has small aberrations other than tilt. In an alternative aspect, the lenslet could be physically altered such that the spot image has a particular structure or profile enhancing its detection with respect to the other spots.

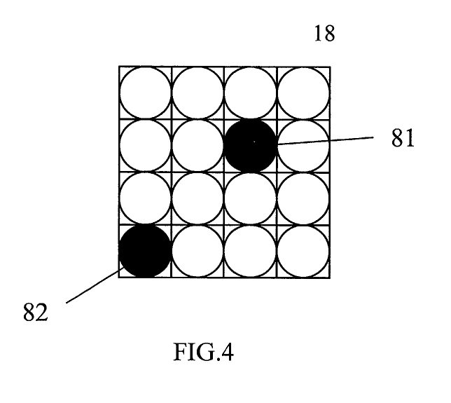

In an alternative preferred embodiment illustrated in FIG. 4, only two, non-adjacent lenslets 81, 82, of the microlens array 18 are optically altered. This offers the benefit of redundancy in the search and identification of the correspondence between spot images and the lenslets. Although implementation of this aspect of the invention will result in the elimination of the respective spot images representing data points for wavefront sampling, adverse effects should not be encountered as long as sampling is sufficient. In addition, as more lenslets are altered, unit costs of microlens arrays will increase due to greater fabrication complexity. There are, however, readily available technologies for producing microlens arrays including lithography, pin-bundle assembly of diamond-turned surfaces, embossing, laser writing, electron-beam, ion-beam and reactive-ion etching, as well as others known to those skilled in the art.

Once all the spot images are correctly identified, analysis of the incoming wavefront is much simplified even when the magnitude of the aberration is increased such as in keratoconic or post LASIK eyes, for example. Thus, the dynamic range and accuracy of the Shack-Hartmann aberrometer are no longer limited by the dimensions and optical properties of the lenslets.

While various advantageous embodiments have been chosen to illustrate the invention, it will be understood by those skilled in the art that changes and modifications can be made therein without departing from the scope of the invention as defined in the appended claims.

I claim:

1. An improved Hartmann-Shack wavefront sensor including a microlens array for imaging a wavefront from an object into an array of spot images on a detector from which an aberration of the wavefront can be determined, the improvement comprising:
a single lenslet only of the array having an alteration that provides an identification of the spot images corresponding, respectively, to the remaining lenslets of the array.

2. The wavefront sensor of claim 1, wherein the single lenslet is a center lenslet of the array.

3. The wavefront sensor of claim 1, wherein the alteration is an optical alteration of the lenslet.

4. The wavefront sensor of claim 3, wherein the optical alteration is a degree of transparency to a wavelength of the object wavefront.

5. The wavefront sensor of claim 4, wherein the lenslet is opaque to the wavelength of the object wavefront.

6. The wavefront sensor of claim 1, wherein the alteration is a physical alteration of the lenslet.

7. The wavefront sensor of claim 6, wherein the physical alteration is an obstruction in at least part of the lenslet.

8. The wavefront sensor of claim 6, wherein the physical alteration comprises the size of the lenslet in relation to the unaltered lenslets.

9. A microlens array for forming an array of spot images of an aberrated optical wavefront comprising a single lenslet only of the array having an alteration that provides an identification of the correspondence between the spot images and the respective lenslets forming the images.

10. The array of claim 9, wherein the identification comprises the positions of the spot images with respect to an anticipated spot image position due to the single, altered lenslet.

11. The array of claim 9, wherein the single, altered lenslet is a central lenslet of the array.

12. The array of claim 9, wherein the alteration comprises a physical alteration of the lenslet.

13. The array of claim 12, wherein the physical alteration is the size of the lenslet.

14. The array of claim 9, wherein the alteration comprises an optical alteration of the lenslet.

15. The array of claim 14, wherein the optical alteration is a degree of transparency of the single, altered lenslet to the wavelength of the aberrated wavefront.

16. A method for improving the dynamic range of a Shack-Hartmann wavefront sensor by enabling the identification of a correspondence between a plurality of spot images of an aberrated wavefront and respective lenslets of a microlens array that form the respective spot images, comprising the steps of:
a) altering a single lenslet only of the microlens array;
b) establishing the presence or absence of a spot image corresponding to the altered lenslet; and
c) from step (b), identifying the remaining spot images corresponding to the respective lenslets that formed the spot images.

17. The method of claim 16, wherein the step of altering a single lenslet only of the microlens array comprises altering an optical characteristic of the single lenslet such that a resulting spot image to be formed by the lenslet is either absent, has a reduced intensity with respect to a spot image formed by a non-altered lenslet, or has an increased intensity with respect to a spot image formed by a non-altered lenslet.

18. The method of claim 16, wherein the step of altering a single lenslet only of the microlens array comprises altering a physical characteristic of the single lenslet such that a resulting spot image to be formed by the lenslet has at least one of a different size and a different shape than a spot image formed by a non-altered lenslet.

19. The method of claim 16, wherein the step of altering a single lenslet only of the microlens array comprises altering a central lenslet of the array.

20. An improved Hartmann-Shack wavefront sensor including a microlens array for imaging a wavefront from an object into an array of spot images on a detector from which an aberration of the wavefront can be determined, the improvement comprising:
an alteration of more than one lenslet but less than all of the lenslets of the array that provide an identification of the spot images corresponding, respectively, to the unaltered lenslets of the array.

21. The wavefront sensor of claim 20 wherein the lenslets are optically altered.

22. The wavefront sensor of claim 20 wherein the lenslets are physically altered.

23. The wavefront sensor of claim 20 wherein only two, non-adjacent lenslets are altered.

24. The wavefront sensor of claim 20 wherein the altered lenslets consist of a sub-array of immediately adjacent lenslets.
